(19) United States
(12) Patent Application Publication
(10) Pub. No.: US 2014/0275848 A1
(43) Pub. Date: Sep. 18, 2014

(54) Patient Monitoring System

(71) Applicant: Nova Technology Corporation, Meredith, NH (US)

(72) Inventor: Michael J.E. Campbell, Meredith, NH (US)

(73) Assignee: Nova Technology Corporation, Meredith, NH (US)

(21) Appl. No.: 14/214,748

(22) Filed: Mar. 15, 2014

Related U.S. Application Data
(60) Provisional application No. 61/791,900, filed on Mar. 15, 2013.

(51) Int. Cl. A61B 5/00

(52) U.S. Cl. CPC A61B 5/0022; A61B 5/0013
USPC 600/301

(57) ABSTRACT

A patient monitoring system including a sensing-recording device that attaches to a patient, a probe utilized to attending personnel at a trauma site and a portable data processing system with wireless communications means with each of said probe and sensing-recording device. A medic utilizes a portable data processing device in the form of a tablet or smart phone to select measurements and to control the operation of the probe and the sensing-recording device. Information obtained from the probe can be transferred to the sensing-recording device. The sensing-recording device includes sensors for continuing to accumulate information about various physiological parameters during the transport of a patient from a trauma site to a medical facility.

### CROSS-REFERENCE TO RELATED APPLICATION

Reference is made to U.S. app. Ser. No. 11/161,688 filed Aug. 12, 2005 for a Patient Monitoring Device and published Feb. 16, 2006 as U.S. Pat. Pub. No. 2006/0036137 which is incorporated by reference in its entirety herein.

This application claims priority from U.S. Provisional Patent Application Ser. No. 61/791,900 filed Mar. 15, 2013 for an Improved Patient Monitoring System which is incorporated by reference in its entirety herein.

### BACKGROUND OF THE INVENTION

1. Field of the Invention

This invention generally relates to monitoring physiological parameters of a patient and more specifically to the monitoring of such parameters in patients who are geographically remote from a medical facility.

2. Description of Related Art

Monitoring of a patient's physiological parameters in a medical environment is an important element of patient diagnosis and treatment. Within a modern medical facility, such as a hospital, such monitoring is relatively easy to perform. The environment is controlled. The staff, including physicians, nurses, physician assistants and technicians, is qualified. Such facilities have equipment resources for enabling the monitoring of one or more patients for a variety of physiological parameters.

Unfortunately the advantages of a hospital environment with its resources and personnel do not exist at many trauma sites, for example, those at typical accident scenes or at battlefield sites. First responders to an accident scene generally include an emergency medical technician (EMT). The EMT assesses the patient and, in conjunction with advice from medical personnel established by radio contact, initiates treatment. Typically the EMT stays with the patient during transport to a medical facility. The EMT can transfer his or her observations to personnel at the facility by interview and/or by written record.

An entirely different situation exists on a battlefield. In a battlefield environment a medic attached to a particular unit treats a wounded patient. When that medic completes treatment at the battlefield site, the wounded patient is transported to another facility. However, the medic stays behind to attend to other patients. The medic's initial treatment protocol will be based on certain readings taken at the site and related procedures for identifying the actions that were taken at the site. For example, if a sedative is administered, the medic may pin the syringe to the patient. Typically no interview occurs between the personnel at the medical facility, such as a field hospital, and the attending medic. Moreover, typically no information is recorded about the patient during initial treatment and transport to a field hospital.

Previous proposals have been made for personal monitoring systems for use in a battlefield environment. For example, U.S. Pat. No. 6,198,394 (2001) to Jacobsen et al. discloses one such system for remote monitoring of personnel. This system utilizes a harness with a number of sensors to be worn continuously by a soldier. The sensors monitor a number of parameters including physiological variables. The harness carries a transmitting unit for transferring data to a central site.

U.S. Pat. No. 6,454,708 (2002) to Ferguson et al. discloses a portable remote patient telemonitoring system using a memory card or a smart card. This system includes a multi-parameter sensor array applied to a patient's chest by means of a sensor band. The smart card or memory card stores measured data. Alternatively, a data logger carried by the patient receives the data. A base station receives the recorded information and transmits it to a remote monitoring site over a telecommunications link. The sensor band is disposable and has a limited life. For long-term monitoring, sensor bands may be replaced periodically.

U.S. Patent Publication No. 2004/0147818 (2004) to Levy et al. discloses a portable system for monitoring and processing patient parameters in multiple operational modes. Specifically a data acquisition processor receives data from sensors on a patient and processes that data. The processed data is communicated to a docking station with a portable monitoring unit in one operational mode or to a network access point in a wireless network in another operational mode.

Each of these proposals requires the use of bulky equipment so it is difficult to maintain an inventory for multiple patients. The Jacobsen et al. patent requires a soldier to wear a harness at all times. First, the soldier must carry extra weight and take precautions to avoid damaging the equipment during normal use. It is not likely that such a harness would be an acceptable alternative for application to a trauma patient on a battlefield or at another trauma site. In addition, medical personnel may have the additional burden of placing the harness on a trauma patient under adverse circumstances.

Each of these systems depends upon the availability of sophisticated communications links, such as those available in a hospital environment. They are not always available at a trauma site or on the battlefield. The use of smart cards or memory cards as suggested in the Ferguson et al. patent present problems because such devices are prone to being lost at a trauma site or during patient transport to a hospital or other central site. Further, only the data measured at the trauma site is recorded.

U.S. Pat. Pub. No. 2006/0036137 to Lewicke and assigned to the same applicant as this invention discloses a patient monitoring system that includes a sensing-recording device that is clipped to a patient and a probe utilized by a medic at a trauma site. After an initial interview, the medic initializes the probe and the sensing-recording device. The sensing-recording device samples one or more physiological parameters for storage in a first memory for the time it is attached to the patient. The probe is reusable and enables the medic to take other measurements, download data from the sensing recording device memory, manipulate the collective data and return data to memory in the sensing recording device. This system allows an EMT to triage patients and ensure an appropriate patient evacuation. The sensing recording device continues to accumulate data in its memory during transport of the patient from a trauma site to a medical facility.

The amount of data about a patient that is conveyed from the trauma site to a medical facility with this patient monitoring system, although increased over information available by prior apparatus, is somewhat limited. For example, when the medic is treating a patient at a trauma site, the medic makes visual observations. However, often time constraints imposed by a particular situation prevent a medic from recording such observations in a form that is easily conveyed to a remote medical facility. Consequently the information resulting from a medic's visual observation is lost because it is not communicated to other medical personnel at a remotely located medical center.

What is needed is a patient monitoring apparatus that enables medical personnel at a trauma site to obtain and record medical information including visual observations, that facilitates the transfer of information about a patient from the trauma site to a remote medical facility and that provides a continuum of data for use by personnel at a medical facility.

### SUMMARY

Therefore it is an object of this invention to provide patient monitoring apparatus that is adapted for use in a variety of environments including battlefield environments.

Another object of this invention is to provide apparatus for continuously monitoring certain physiological parameters of a patient from the arrival of a first responder or medic at a site to the arrival of the patient at a medical facility.

Yet another object of this invention is to provide economical patient monitoring apparatus that is easy to use and facilitates the measurement of physiological parameters and enables the recording of visual observations for transfer to a medical facility

In accordance with one aspect of this invention, apparatus for monitoring a patient includes three basic elements, namely: a probe, a sensing-recording device and a computer-based tablet or equivalent device. The tablet includes application programs available to the medical personnel at the trauma site. Wireless communications establish communications with the tablet, the probe and the sensing-recording device.

### BRIEF DESCRIPTION OF THE DRAWINGS

The appended claims particularly point out and distinctly claim the subject matter of this invention. The various objects, advantages and novel features of this invention will be more fully apparent from a reading of the following detailed description in conjunction with the accompanying drawings in which like reference numerals refer to like parts, and in which:

FIG. 1 is a block diagram of patient monitoring apparatus that incorporates this invention; and

FIG. 2 is a flow chart that is useful in understanding the operation of the apparatus in FIG. 1.

### DESCRIPTION OF ILLUSTRATIVE EMBODIMENT

FIG. 1 discloses a patient monitoring apparatus 10 with a separate sensor button-like device that records data related to at least one medical parameter, hereinafter a “sensing-recording device 11” and a portable probe 12. In use, a medic attaches a single sensing-recording device 11 to a single patient from an inventory carried by the medic. In the following discussion the term “medic” normally identifies military personnel; however, in conjunction with this invention the term includes both military personnel and civilian personnel such as an EMT. Each medic will also have a single probe 12 which will interact with any proximate sensing-recording device, such as the sensing-recording device 11.

As described in the above-identified U.S. Pat. Pub. 2006/0036137, the sensing-recording device 11 includes a base unit 13, a clip having a transfer portion and a clip arm 15 spaced and essentially parallel to the housing 13. The housing 13 also carries an on/off button 16 and an interface and wireless communications module 17 that typically is located within the housing 13. Different communication protocols can be utilized including Bluetooth® technology. The sensing-recording device 11 can include a dermal phase meter (DPM) electrode assembly, a temperature sensor and a light source and complementary photocell. These sensors provide moisture measurements, patient temperature measurements and pulse oximetry measurements. Other sensors that provide other measurements could augment or replace the above-identified sensors.

The probe 12 in FIG. 1 has a housing 20. An end portion 21 carries an electrode assembly 22 that is constituted by a probe DPM electrode assembly which may also include temperature and pressure sensors or monitors. In addition the probe 12 may contain a sensor for monitoring mean arterial blood pressure or other physiological parameters. The probe additionally includes an interface and wireless communications module 23.

FIG. 1 also depicts a portable data processing system 40 that can be constituted by a wireless tablet (e.g. an Apple® iPad® tablet or Android® or like devices) or a smartphone (e.g. Apple iPhone® or Android mobile telephones or the like devices) that allows the diagnostician to interact with both the sensing-recording device 11 and the probe 12 through a wireless communications module 41, control and applications programs 42, a memory 43, a data input\output mechanism 44 and a visual output 45 such as a tablet or smartphone screen. This provides wireless communications and establishes control between the tablet 40 and the probe 12 through the interface of wireless communications module 23 and between the tablet 40 and the sensing-recording device 11 through the interface and wireless communications module 17. Although not shown, it will also be obvious that the tablet 40 will also have the capability of transmitting and receiving information over wireless networks such as the Internet and have an imaging capability.

Now referring to FIGS. 1 and 2, in one implementation of this invention a typical medical measurement session begins when a medic interacts with the tablet 40 to initiate a measurement operation in step 50 of FIG. 2. At this point, the medic can enter patient information into the tablet 40 along with other relevant information. If the sensing-recording devices are bar-coded or include other unique identification codes or the like, the medic can use the tablet 40 to record that identification directly. The medic performs these and other operations by interacting with the tablet 40 in response to an application program. At step 51 the medic enters any environmental parameter values that are required concerning the environment in which the medic is operating. For example, the medic could enter the current ambient temperature and relative humidity.

Assuming the medic has applied the sensing-recording device to the patient and turned it on, step 52 enables communications between the tablet 40 and the sensing-recording device 11 through the wireless communications modules 41 and 17 respectively. When this occurs, step 53 can record the sensing-recording device 11 identification, if not previously recorded, and can create a log file for recording of data in the sensing-recording device memory in step 54.

While this occurs, the medic can use step 55 to photograph the patient with the tablet 40 and record each photograph. Such photographs might include close-ups of injury sites. This is advantageous when a patient suffers from burns, particularly when an imaging device has a photo-temperature capability.

At step 56 the medic, through interaction with the tablet 40, enables communications with the probe 12. In step 57 the medic takes probe measurements and those measurements are relayed back to the tablet 40 for storage in the memory 43. Software will combine collected data with the respective digital photographs for later transmission to a hospital.

At step 60 the application program formats the probe measurement data and sensing-recording device data for output. There are several output alternatives. FIG. 2 discloses an e-mail option. If selected, step 61 transfers control to step 62 whereupon the tablet e-mails the patient data, including any photographs, to a patient destination such as a battlefield hospital or civilian medical facility. If the memory in the sensing-recording device 11 has sufficient capacity, step 63 could divert control to step 64 thereby to download all the data, including any photographic data, to the sensing-recording device 11. In this event, the sensing-recording device 11 has all the information pertaining to the patient from both the sensing-recording device 11 and the probe 12. This combined data may be downloaded at a battlefield or civilian facility that receives the patient. At that point, the operation with a particular patient would be complete.

This invention has been disclosed in terms of certain embodiments. As will be apparent, a specific implementation of the measurement system 10 can take many forms that are well within the purview of the persons of ordinary skill in the art. FIG. 1 depicts a specific dermal phase meter probe and sensing-recording device. Other configurations could be substituted for measuring the same or other parameters. Other wireless tablets, mobile telephones and like devices could be substituted for the portable data processing system 40. An independent imaging system could be used in lieu of, or could be used as a complement to, the imaging system in the wireless tablet or mobile telephone. Different functions could be performed in different components. For example, in one embodiment, the probe preprocesses measurement data for transfer to the tablet. Alternatively, the tablet could receive the raw measurement data for preprocessing in the tablet. Thus it will be apparent that many modifications can be made to the disclosed apparatus without departing from the invention. Therefore, it is the intent of the appended claims to cover all such variations and modifications as come within the true spirit and scope of this invention.

What is claimed as new and desired to be secured by Letters Patent of the United States is:

1. A system for monitoring a patient a patient comprising:
A) a portable probe for measuring various physiological parameters in response to measurement commands and for generating information including the outcome of the various physiological parameter measurements commands,
B) a sensing-recording device for attachment to a patient for measuring various physiological parameters and for generating information including the outcome of such measurements,
C) a portable data processing system for generating the measurement commands and receiving measurement information generated by said probe and said sensing-recording device, and
D) wireless communication means in each of said portable probe, sensing-recording device and portable data processing system for interconnecting said probe, said sensing-recording device and said portable data processing system whereby the physiological parameter measurements can be displayed at said portable data processing system.

2. The system as recited in claim 1 wherein said portable data processing system includes said portable data processing system and said wireless communication means include means for transmitting measurement information to a remote location.

3. The system as recited in claim 1 wherein said portable data processing system additional includes imaging means for imaging patient conditions and means for conveying to a remote facility the measurement information and the images from said imaging means.

4. The system as recited in claim 1 wherein said portable probe includes at least one sensor for physiological parameters and said portable data processing system includes means for transferring information to said sensing-recording device.

5. The system as recited in claim 1 wherein said portable data processing system includes means for receiving environmental parameters from said portable probe.

6. The system as recited in claim 1 wherein said portable data processing system includes a visual display and means for displaying measurement data on said visual display.

7. The system as recited in claim 1 wherein said portable data processing system and said wireless communications means include means for transferring patient information to a remote location.

8. The system as recited in claim 1 wherein said portable data processing system and said wireless communications means includes means for transferring information to said sensing-recording device.
